(19) United States
(12) Patent Application Publication
(10) Pub. No.: US 2020/0066399 A1
(43) Pub. Date: Feb. 27, 2020

(54) AN IMPROVED PROCESS AND SYSTEM FOR MANAGING HEALTHCARE NETWORK PERFORMANCE

(71) Applicant: CITIUSTECH HEALTHCARE TECHNOLOGY PRIVATE LIMITED, Mumbai (IN)

(72) Inventors: Jeffrey Springer, Philadelphia, PA (US); Shailendra Rao, Jogni (IN); Anvesh Patel, Ahmedabad, Gujarat (IN); Pranjali Joshi, Mumbai (IN)

(21) Appl. No.: 16/611,762

(22) PCT Filed: Nov. 13, 2018
(86) PCT No.: PCT/IB2018/058887

§ 371 Date: Nov. 7, 2019

(30) Foreign Application Priority Data
Nov. 13, 2017 (IN) 201721040339

(51) Int. Cl. G16H 40/20; G06Q 10/06; G06Q 10/10

(52) U.S. Cl. CPC G16H 40/20; G16H 10/60; G06Q 10/109; G06Q 10/06393

(57) ABSTRACT

The present invention relates to the field of healthcare and particularly relates to an unproved process and system for collaboration between various stakeholders in a healthcare industry to ensure that a key performance indicator correctly identified and computed, and any opportunity, which causes low performance on the key performance indicator is communicated to all stakeholders to ensure required services are completed and codified to ensure proper tracking to addressing of the said opportunity, the method comprising: identifying beneficiary's data or information from various sources across a healthcare network (131); defining a key performance indicator (KPI) library for setting live rules to process the incorporated data or information of the beneficiary (132); providing feedback of the completed KPIs (137); examining and identifying whether beneficiary requires further opportunity for improvement in healthcare services (133); reviewing the opportunity and enabling appropriate stakeholders to engage with the beneficiary in the appropriate way to address the identified opportunity (201) for beneficiaries that requite care, notifying the patients to schedule them either through direct outreach or through electronic notification so the beneficiary can schedule their own appointment (204/205); reviewing the identified opportunity during the encounter/visit and addressing tire identified opportunity (206); capturing the opportunity has been eared (209); further care is needed (208); the care has already been provided or there is an exclusion or exception that means the care is not needed for the said beneficiary (No number on chart); charting the captured information in the proper manner to address the opportunity in the KPI to provide the care to the beneficiary (212); coding the charted information in the proper manner to ensure that the opportunities in the KPIs are addressed properly (213) for reporting systems for providers to get credit for services rendered, re-evaluating the captured information to determine whether the data created in the coding and charting completed the intent of the KPIs (217); ascertaining the care provided and marking the KPIs as addressed in analytics by using an enriched claim to communicate the proper information to address the opportunity in the KPIs (215); and updating the information of the beneficiary to the analytic system and payer system (216).

### FIELD OF THE INVENTION

The present invention relates to the field of healthcare and more particularly to an improved process and system to allow efficient collaboration between various stakeholders in healthcare industry, such as but not limited to providers, payers and ancillary service providers, and beneficiaries to enable low cost and real-time identification of opportunities for improvement in quality of healthcare services delivered to a beneficiary (i.e. patient).

### BACKGROUND OF THE INVENTION

Stakeholders in the healthcare industry have defined standards of care in order to increase efficiency in the industry and to provide an improved level of healthcare to the general population. The Key Performance Indicators (KPIs) of healthcare network performance and quality of care include regulatory clinical quality measures, Diagnosis, Cohorts, Protocols, contractual quality measures, operational quality measures, financial measures, risk stratification methods (example Hierarchical Conditional Coding), as well as the particular actions such as recommended or required screenings, tests and treatments, standard care guideless etc. For example, clinical quality measures are well defined KPIs to determine whether appropriate care was delivered and are usually calculated using healthcare data such as both clinical and financial data, including coding and clinical results and tests. This information often needs to be shared with other healthcare organizations (such as payer, ACOS, CINs, IPNs and IPAs). Standards of care for each individual are based on individual's demographics and medical conditions. The standards of care may also define certain time frames and/or locations that are recommended or required for the particular actions of care delivery to take place, and may define which of the healthcare stakeholders should perform the particular actions, various standards of care include different lists of predefined opportunity in care to help stakeholders efficiently identify and prioritize recommended or required actions to be completed to optimize the delivery of care to an individual or population. Opportunity refers to the deviation in delivering care to a patient. Further, the opportunity includes a gap in clinical quality, assessment and/or stratification, diagnosis, care, screening, tests, treatments, assessment, documentation, waste avoidance, data accuracy, compliance etc.

In the modern era of health care, most providers have adopted electronic health records (EHR) that document the electronic health record of beneficiary and services provided. The provider, service provider or healthcare provider is an individual who provides preventive, curative, promotional or rehabilitative health care services in a systematic way to people, families or communities. These are effective tools for recording historical information that was once charted on paper, and most EHRs can offer directions and reminders that may help bridge opportunity in care. However, these systems are ill suited for closing the health care opportunity among providers and fully supporting the coordination of care across all health and social services. Health systems recognize the importance of integrating physical, behavioural, and social determents of health, new technology solutions must be adopted to support care among providers and stakeholders in healthcare industry.

Actual care is provided at the time of visit to various places, care settings, where care is delivered such as Physician Offices, Clinics, Hospital etc. Thus, at the time of visit the healthcare provider is burdened to manage an enormous amount of information in order to identify all opportunities in care for each of their beneficiary, i.e. person seeking care, at the appropriate time. Traditional methods do not place prime focus on addressing opportunities or are able to address opportunities only partially because of limited set of data. Also, traditional healthcare systems do not ensure that gaps are closed within the existing workflows of the healthcare service providers and payers (who pay for service provided by healthcare service provider). Often the opportunities are identified months after the patient leaves the providers office.

The contemporary processes of closing gaps are time consuming, costly and inefficient processes which are not scalable for large population of patients or health plan members. The contemporary process either requires a tremendous amount of time and focus by providers that takes them away from seeing patients or involves retrospective review of the data to determine which patients should visit the provider or has additional gaps. This often requires data collection/exchange through a manual process, commonly known as chart chase or charting, wherein one healthcare organization (such as payer) reaches out to another healthcare organization (such as a provider) through mail, email, phone, fax, in person visits etc. to gather detailed healthcare information on one or more individuals or through new processes driven by care management or charter to engage the patient outside their visit. Highly-skilled and high cost professionals, specially nurses, end up spending large amount of time in interpreting the requirements of this data collection and facilitating it. This time can be used more efficiently if physicians are able to focus on treating patients and the identification of the data needed to close the gaps is done within the normal data exchange processes, such as claims coding and processing, that already exist in traditional healthcare systems. Another drawback of the contemporary method is the long-time gap, usually in the timeframe of weeks or months, between identification and closure of gaps. Another drawback of the contemporary method is the high cost of reactive gap identification and gap closure through contemporary processes including but not limited to medical record retrieval (MRR), chart chases, chart abstraction, population health dashboards etc.

In order to address the problems discussed above, the present invention aims to provide a new process which identifies the opportunity to deliver care at the time of patient visit. Opportunity refers to the deviation in delivering care to a patient. Further, the opportunity includes a gap in clinical quality, assessment and/or stratification, diagnosis, care, screening, tests, treatments, assessment, documentation, waste avoidance, data accuracy, compliance etc. The present invention comprises of multiples data sets for gathering information of the patient. Thus, the present invention enables healthcare provider to indicate which opportunity was addressed in the course of providing care and include the addressed opportunity with the claim and clinical documentation created by a coder and charter to ensure the accurate tracking of action in the existing process rather than creating a separate follow-up to ensure that the opportunities are properly addressed. For addressing opportunity of care that require follow-up after the coding process, the invention will drive workflow appropriately to either the physician or any other stakeholder, a follow-up appointment or tracking additional care is completed in appropriate amounts of time. This process increases quality of healthcare delivery while controlling the cost involved in it.

### SUMMARY AND OBJECTS OF THE INVENTION

A primary object of the present invention is to improve overall healthcare network performance and improve the quality of healthcare services and ensure that adequate care is delivered by creating a closed loop system for tracking and documenting care completion throughout their lifecycle.

Another object of the present invention is to include multiple data sets for tracking updated records of beneficiary (i.e. a patient) with more metric(s).

Another object of the present invention is to efficiently provide opportunity for delivering care to the beneficiary.

Another object of the present invention is to provide more key performance indicators (KPI) with pre-built rules as well as flexibility to create and/or modify the rules based on the measured performance of the service provider.

Another object of the present invention is to reduce time and costs of monitoring and improve quality of healthcare services.

Another object of the present invention is to build scalable processes of monitoring and improving quality of healthcare for large populations of patients.

Another object of the present invention is to provide an improved process to identify the opportunity to deliver care at the time of visit, enable simple documentation by the provider of addressing the opportunity and compare the delivered opportunity with the documentation (claims and clinical documentation) needed to ensure the accurate tracking within the existing process rather than creating a separate follow-up so organizations can ensure opportunities are properly addressed. The new process will create and/or capture additional data which can be used by stakeholders, involved in delivering care, to document the addressing of opportunity. This is often clinical documentation that is not included in a claim to ensure successfully providing the opportunity of care to the beneficiary.

Another object of the present invention is to provide an improved process which allows for improved quality of healthcare reducing time and costs by presenting accurate and right amount of information to care giver, such as physicians, during a patient visit so that they can identify opportunities that they have closed in the visit and then ensure the proper documentation is created in the coding and charting after the visit or encounter.

A further object of the present invention is to provide an improved process that takes administrative burden off highly-skilled, high cost professionals such as physicians and, thus allow them to focus on their core responsibility of treating patients.

Another objective of the present invention is to track the addressing of opportunity(s) after the visit for follow-ups that should occur and ensure their completion in a timely manner. Enabling notification of correct stakeholders if the correct actions have not been taken.

Yet another object of the present invention is to provide an improved process which allows interpretation and automated flow of healthcare information in the normal exchange of records between various stakeholders such as health plans, care givers, physicians, lab services etc improving the quality of healthcare services.

Accordingly, a first aspect of the invention relates to a new process which helps in identifying the opportunity in care and communicating them to healthcare service providers so that they can take appropriate action such as delivering a certain type of care or ordering a new test. Then charter documents delivered care as per charting guidelines. Then the coders will enable the coding process to compare the coded data with the data to be coded provided by the healthcare service provider or charter, and accordingly document codes based on the action taken by the healthcare service provider or charter to ensure that the accurate tracking of action in the existing process is correct and the process will create or capture additional data to communicate the appropriate information to indicate opportunity have been addressed rather than creating a separate retrospective follow-up so organizations can ensure opportunities are properly addressed and to successfully provide the opportunity of care to the beneficiary.

The process of the invention allows for improving quality of delivered healthcare services, reducing both time and cost of identifying opportunity of care information in the most simplistic way possible at the time of visit itself (e.g. during patient visit to physician) and to the coder, to ensure that all the care provided is documented in the claim and additional supplemental data sent to stakeholders. The process also enables coders and administrators to test and validate the opportunity that have been addressed by physicians during a visit and are closed in documentation claims and appropriate attachments are updated accordingly. For opportunity that have not been addressed, workflow to track appropriate work enables transparency and appropriate follow-ups. The process further enables improved quality of healthcare services by freeing up bandwidth of highly skilled and high-cost professionals such as physicians and transferring the administrative burden to other relatively lower cost team members such as medical coders, medical assistants, etc. The improved process also automates transfer of healthcare information between various stakeholder healthcare organizations such as health plans and providers, thereby providing improved scalable processes of monitoring and improving quality of healthcare for large populations of patients.

The process is implemented by or on a computer, server, workstation, system, or other device. A memory stores information. A processor, executing instructions, performs the acts, such as mining, causing storage, converting, evaluating, identifying, and outputting.

Other devices may be used with or by the processor, such as a user input and display being used for editing and/or other activity.

Accordingly, a method for collaboration between various stakeholders in healthcare industry to correctly identify and compute key performance indicators (KPIs), and communicating any opportunity, which increases performance on said KPIs, to all stakeholders, and ensuring identification, codification and completion of required services addressing said opportunity, the method comprising the steps of: identifying beneficiary data or information from various sources across healthcare network; defining KPIs for setting the rules to process the incorporated data or information of the beneficiary for identification of an opportunity; computing the performance based on said KPIs (137) against the data collected on the beneficiary; examining and identifying whether there exists any opportunity for improvement in delivering healthcare services to beneficiary; reviewing the identified opportunity and enabling appropriate stakeholder to engage with the beneficiary in the appropriate way to addressed the opportunity; reviewing the identified opportunity and enabling appropriate stakeholders to engage with the beneficiary in the appropriate way to close the gap; for beneficiaries that require care, notifying them through direct outreach or through electronic notification so that the beneficiary can schedule their own appointment; reviewing the identified opportunity(s) when the beneficiary comes for care during an encounter/visit of the beneficiary and capturing that the opportunity has been addressed, further care is needed, the care has already been provided or there is an exclusion or exception that means the care is not needed for the said beneficiary; charting said captured information in a proper manner to address the identified opportunity as per the defined KPI to provide the care to the beneficiary; coding the charted information in a proper manner to ensure that said opportunities as per the defined KPIs are addressed properly; re-evaluating the captured information to determine whether the data created in the coding and charting completed the intent of the KPIs or a follow up visit/encounter is needed with beneficiary; ascertaining the care provided and marking the KPIs and opportunities as addressed; and updating the information of the beneficiary to the analytic system and payer system.

The step of ascertaining the addressing an opportunity of care includes creating additional records to submit where a claim does not support the ability to capture data needed to delivering opportunity as a modification to existing claim format, or adding an additional data exchange format hardcopy or electronic-copy to existing claim format, or replacing the existing claim format by some other data exchange format hardcopy or electronic-copy.

Another aspect of the invention relates to a system for addressing an opportunity in healthcare services. The system for collaboration between various stakeholders in healthcare industry, such as but not limited to providers, payers and ancillary service providers, and beneficiaries to enable low cost identification and addressing an opportunity in quality of healthcare services delivered to a beneficiary i.e. patient includes a network of computers, storage, graphical user interface and servers and one or more of backend information systems, modules etc. One or more of these systems may be non-local and may be accessed over a wide-area network (WAN) and/or the internet.

The aspects of the present disclosure may be embodied as a system, method, or computer program product. Accordingly, aspects of the present disclosure may take the form of an entirely hardware embodiment, an entirely software embodiment (including firmware, resident software, micro-code, etc.), or an embodiment combining software and hardware aspects that may generally be referred to herein as a module or system.

It is to be understood that the aspects of the present disclosure, as generally described herein, can be arranged, substituted, combined, separated, and designed in a wide variety of different configurations, all of which are explicitly contemplated herein.

Furthermore, aspects of the present disclosure may take the form of a computer program product embodied in one or more non-transitory computer readable medium(s) having computer readable program code encoded thereon.

Any combination of one or more non-transitory computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. In the context of this document, a computer readable storage medium may be any tangible medium that can contain or store a program for use by or in connection with an instruction execution system, apparatus or device.

Any one or more of the aspects described above may be used alone or in combination.

### BRIEF DESCRIPTION OF THE ACCOMPANYING DRAWINGS

The foregoing and other objects, features, and advantages of the invention will be apparent from the following detailed description taken in conjunction with the accompanying drawings, wherein:

FIG. 1 illustrates an exemplary process for providing opportunity of care in healthcare system according to the present invention;

FIG. 2 illustrates by means of an exemplary process flow diagram the overall process for addressing opportunity(s) of care in healthcare system according to the present invention.

### DETAILED DESCRIPTION OF THE INVENTION

Embodiments of the presently disclosed invention will now be described in detail with reference to the drawings. The disclosed system and method automatically identify opportunity(s) to provide services in care of individual patients, enables providers to view the identified opportunity of care and to take efficient action to address those opportunities.

As used in this specification, real-time refers to an action that occurs contemporaneously or nearly contemporaneously (e.g., within several seconds of) with another event.

As used in this specification, a healthcare opportunity or opportunity or opportunities or opportunities in quality of healthcare refer to deviations, in delivering care to a beneficiary i.e. patients, from industry standard or commonly accepted guidelines of providing care and can be identified through mechanisms including, but not limited to, processing of clinical quality measures, financial metrics, operational metrics, risk assessment (example Hierarchical Condition Coding) evidence based care guidelines, clinical decision support rules etc. For example, a healthcare opportunity may refer to a gap in clinical quality, diagnosis, care, screening, tests, treatments, assessment, documentation, waste avoidance, data accuracy, compliance etc. It should be understood that the change of healthcare state and the updated status may be based on changes in a patient's health parameters but may also be based on other parameters such as record keeping parameters in which gaps may include incorrect coding or an incomplete chart in a patient's health record or gaps in a claims submission, or other parameters such as the timeframe and the underlying data being considered to evaluate presence of gap. For example, the gaps in care may also include gaps in risk adjusted conditions, gaps in screening and counselling and gaps in clinical quality and preventive care, medications etc. The first set of rules that define gaps in care may include identifying inconsistent information in the healthcare information of the patient as a gap in care of the patient.

As used in this specification, a healthcare facility, healthcare industry, healthcare network or healthcare service providing institutions includes physician's offices, hospitals, clinics, ambulatory surgical centres, emergency care departments, or other locations or organizations for treating illness or injury. The patient may stay for a part of a day or for one or more days at a healthcare facility for diagnosis and/or treatment herein referred to as a visit or encounter. In some cases, the visit may be only minutes. The care of the patient before, during, and after any visit/encounter at a healthcare facility or physician's office is managed. By identifying an opportunity in care, the treatment and/or diagnosis may be improved, the processes at a healthcare facility or physician's office may be improved, and/or quality of care may be determined.

As used in this specification, a payer or healthcare payer, generally refers to entities other than the patient that finance or reimburse the cost of health services. In most cases, this term refers to insurance carriers, other third-party payers, or health plan sponsors (employers or unions) etc.

As used in this specification, a provider, service provider or healthcare provider is an individual who provides preventive, curative, promotional or rehabilitative health care services in a systematic way to people, families or communities. A provider may operate within all branches of health care including medicine, surgery, dentistry, midwifery, pharmacy, psychology, nursing or allied health professions etc.

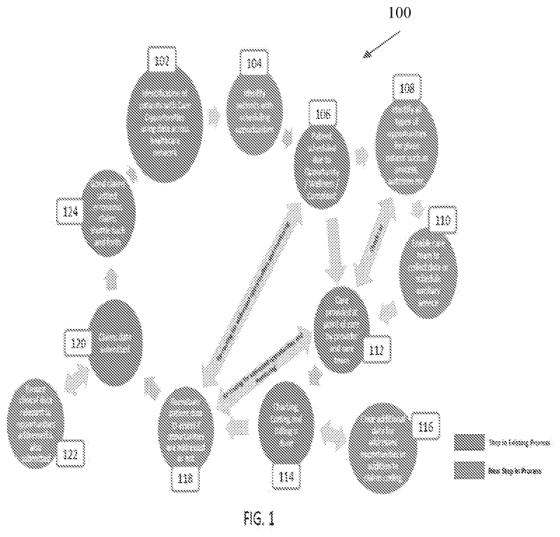

Referring to FIG. 1, a process 100 is illustrated for addressing an opportunity in healthcare services according to the present invention. The process 100 starts with identification of beneficiary using or gathering data from various sources across healthcare system in step 102. For example, data may be obtained from payers, providers, laboratories, imaging, hospital systems, claims data or the like. Beneficiary can also encounter/visit a service provider because they fell sick or for a wellness visit and not because they were scheduled for an encounter/visit because of the identified opportunity. Such beneficiary is also examined for identifying the opportunity for services in medical care. The beneficiary with opportunities are identified in step 104. Identified beneficiary having one or more addressed opportunities are notified and an appointment with a service provider and a care team is scheduled in step 106. Beneficiary encounter/visit can also happen for patients not identified under step 104 for various other reasons such as patient proactively visiting provider due to sickness, for a wellness visit, regular consultation etc. and the steps of this new process will also apply to such beneficiary and for those beneficiaries who visiting first to the service provider. At step 108 all types of the opportunities for given beneficiary is identified and a check list of such identified opportunity is provided to the care team. The identified one or more opportunities enable the care team at step 110 to collect data relating to such opportunities or schedule ancillary services to addressing the opportunity. Care team provides information on opportunity in beneficiary's care and enables providers and medical assistants to substantiate their findings and upload necessary supporting documentation. The care team also identifies risk adjustment related opportunity such as suspected conditions, conditions that have been documented in the past, or screenings that are due to be performed. When the patients arrive for an appointment at step 112, they are checked, and care provided at point of care by provider and care team. Step 112 may include review of opportunities in care, and the beneficiary has the option to voluntarily disclose any opportunities in care that may have already been addressed or kept pending. At the end of the appointment at step 114, charting, coding and billing is done. In addition to the claims coding, the identified additional data for addressing an opportunity is coded in step 116, in existing file formats, or modified versions of existing file formats, or by introducing additional file formats, new or existing, to currently used claims files, here onwards referred to as enriched claims data or claim data or claim. The data or information being coded in the process is not limited to existing file formats only but can be coded in new ones or modified file formats or encompasses all other existing and/or new file formats which may be developed in due course. The collected beneficiary data is revaluated at step 118 to assess if opportunity has been addressed or not. If it is found that the opportunities relating to the beneficiary is not addressed the entire step from 106 to 114 is repeated. The beneficiary is re-routed for another appointment as in step 106 with the care team and all the identified opportunities are attended to by the care team as in step 112 and the opportunities are provided. Once the opportunity has been provided ascertained in step 118 and based thereon an enriched claim data is submitted for evaluation at step 120 together with data or documents evidencing for providing opportunities as shown in step 122. When a claim received contains diagnostic code(s) and/or procedure code(s) and/or medications that indicate that an opportunity in care is addressed, for the patient mentioned on the claim. The submitted claim data is evaluated at step 124 and good claims are settled and erroneous claims shuttle back and forth between the service provider of healthcare service (entity who incurred cost) and payer of these healthcare costs.

Between different beneficiary found with opportunities in care, there is no ordering implied on the steps to be performed. For example, it is not necessary to complete the check ins for all beneficiaries with opportunities in care before executing the check out for any patient. It is not intended in the illustration.

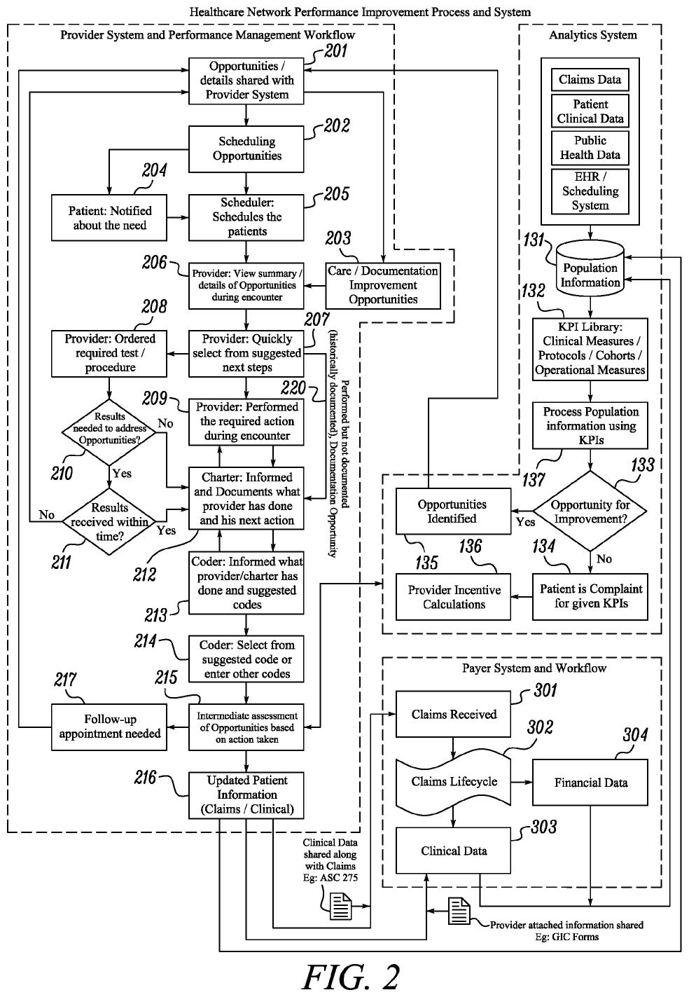

Referring now to FIG. 2, a method for collaboration between various stakeholders in a healthcare industry to correctly identify and compute key performance indicators (KPIs), and any opportunity, which increases performance on said KPIs communicated to all stakeholders ensuring identification, codification and completion of required services addressing said opportunity the entire method is processed by three different systems. The first system is an analytics system which comprises a central database server that hosts all the data, including but not limited to clinical, claims, risk and others, about the patient. It receives information from multiple sources, transforms, reconciles and stores the information for processing. The central database server also hosts a Rules Management Module (RMM) which is an intuitive interface which allows creation of KPIs easily. RMM helps in improvement of turnaround time of creating new, customizing existing and implementing KPIs such as clinical quality measures, hierarchal condition coding, cohorts and other types of KPIs described earlier. Clinical quality measures (CQMs) are tools that help measure and track the quality of health care services that eligible professionals (EPs), eligible hospitals, and critical access hospitals (CAHs). CQMs could be defined by a number of regulatory bodies including by not limited to NCQA, CMS, ONC, State Medicaid programs and Official Registries, contractual measures which are defined as individual contracts between payers and providers to drive quality improvement, operational measures defined to create consistent care, Risk Adjustment Measures defined to categorize a patients list of comorbidities, Clinical Decision Support measures defined to create alerts on the process of care for patients and Cohorts which is used to define a group of patients who have similar comorbidities, which may be monitored over a period of time. The analytics system is powered by BI-Clinical Rules engine with uses rules defined in RMM and data from database to produce compliance statues.

The next system is service provider system, these are mainly electronic health records (EHR) or electronic medical records (EMR) with or without traditional Network Performance Improvement Workflow. This system empowers provider practices during patient encounters.

The last system is payer system which usually receives the claims data from various system which includes service provider systems or data analytics system or the like.

The process for identifying and providing opportunity of care in healthcare system will begin when a beneficiary will encounter/visit the healthcare service provider who is registered with the analytics system. The analytics system compiles set of data which comprises claims data, patient clinical data, public health data and electronic health record (EHR) or scheduling data of the patient. When the beneficiary encounter/visits the healthcare service provider, the service provider checks the history of the beneficiary from the analytics system by using the client device. The client device may include one or more processing devices, and may be, or include, a mobile telephone (e.g., a smartphone), a laptop computer, a handheld computer, a tablet computer, a network appliance, or a combination of any two or more of these data processing devices or other data processing devices. Alternatively, a mobile device associated with the beneficiary may detect, e.g., using a global positioning system (GPS) sensor, a Wi-Fi sensor, or a beacon detector, that its location matches a location that is associated with the location of the doctor, thereby triggering the beneficiary encounter/visit notification.

The client device can communicate with the service provider which may include for example a cloud-based system, anything as a service (XaaS) system, a standard server, a group of such servers, or a rack server system, and may be accessed via any network such as the wired or wireless local area network (LAN), wide area network (WAN).

Those beneficiaries who have are not explicitly scheduled to visit can also visit a service provider because they fell sick or for a wellness visit. Such beneficiary is also examined based on the information provider by the electronic health records EHR/electronic medical records EMR or public health data for identifying the opportunities and to take action to provide the identified opportunities at beneficiary visits.

Referring to FIG. 2 an exemplary process flow diagram for collaboration between various stakeholders in a healthcare industry to correctly identify and compute key performance indicators (KPIs), and any opportunity, which increases performance on said KPIs communicated to all stakeholders ensuring identification, codification and completion of required services addressing said opportunity according to the present invention, the healthcare provider system may access the population information system 131 of the analytic system to obtain the identified opportunity. The population information system 131 compile claims data, beneficiary clinical data, public health data and electronic health record (EHR) or scheduling data of the beneficiary or the like. The population system 131 further process the data to the key performance indicator (KPI) library. This KPI library define rules or protocols for Clinical Quality Measures, Diagnosis, Cohorts, Protocols, Contractual Measures, Financial Measures, Operational Measures, Risk Adjustment Measures or the like to process the incorporated data or information of the beneficiary for identification of an opportunity. These rules or set of protocol define the prescribed time in which the opportunity has to be addressed to the beneficiary. Once the rules or standard set of protocols are defined by the KPI library 132 the population information system processed the incorporated data or information of the beneficiary. At step 137 the performance is computed on said KPIs against the data collected of the beneficiary at step 131. At step 133 the data analytics system checks whether the beneficiary required further opportunity for improvement in the provided services. If yes, then the identified opportunity 135 is shared with the service provider system. If no opportunity exists on a KPI and then beneficiary is compliant 134. Provider incentive may optionally be calculated as an additional step 136.

Now, the process moves to the provider system where an identified opportunity of the beneficiary is shared with the provider system. The service provider system reviewing the identified opportunity and enabling appropriate stakeholders to engage with the beneficiary in the appropriate way to address the opportunity at step 201. Based on the identified opportunity the service provider system decides how to examine this opportunity by scheduling the appointment by the beneficiary at step 202 or if care will be provided without scheduling appointments by further providing improvement in opportunities at step 203.

At step 204 beneficiary is notified about the need to address identified opportunity as per the clinic's protocol and in terms of scheduling, process, outcome etc. The clinical module/system provides information in such a way that more opportunity and/or all the opportunity relating to the identified beneficiary can be addressed in one scheduled encounter/visit. Then the scheduler schedules the appointment of the beneficiary with the provider for addressing an opportunity at step 205 wherein a service provider system uses all the demographic and clinical data available to identify the opportunity in care for beneficiary. The identification is done by running the beneficiary that are scheduled against all potential clinical quality measures and using that to identify any opportunity in care. The notification of the schedule to the beneficiary is either through direct outreach or through electronic notification so the beneficiary can schedule their own appointment. Accordingly, a list of beneficiaries to be contacted is created. The identified opportunities which required further improvement in care and the opportunities which identified by beneficiary encounter/visit are forwarded to the provider at step 206 where the provider review and examine the details of the opportunities which are encountered at step 206.

For the identified beneficiary, it is checked and assessed whether all the identified and additional opportunities can be addressed in the scheduled visit of the beneficiary at step 206. The information available at step 206 relating to a beneficiary is further examined at step 207 to provide easy view to healthcare provider into care guidelines for the said beneficiary. Tracking the captured opportunity to be addressed against a workflow which tracks orders or tests and informs appropriate stakeholders whether the additional procedures, medications or tests performed or not performed in appropriate timeframes (208) for addressing an opportunity. The service provider suggests at step 209, what the next action required to address the encountered opportunity at step 207.

The service provider reviews all the available information relating to the beneficiary so that they are enabled to mark the beneficiary record with the facts/updates, for example, address opportunity of care during the visit, ordered a test or procedure or mark the opportunity as not valid due to pre-existing conditions or exclusions and define next steps at step 208. The ordered test can either be driven by a person (i.e. scheduling a beneficiary for a follow-up) or for system tracking of progress on ordered procedures (for example, colonoscopy) or test (i.e. labs). The care that has already been provided or there is an exclusion or exception that means the care is not needed for the said beneficiary. The results of the ordered test that are generated are evaluated to determine if they address an opportunity at step 210. If the results do not address an opportunity directly, then the result will forward to the charter for additional documentation at step 212. If the results do address an opportunity, then the results will be determined if they were received in a prescribed time limit at step 211 and if they were, the received results will be forward to the charter for documentation. If the results are not received within time limit, then the results will forward to the provider system 201 for finding the lack of opportunity for delivering the results.

Once all the available and additional data is captured it is ensured at step 212 that all care provided by the provider is documented and recorded and the documentation/charting relating to the opportunities has been done. Charter compare the already documented data with the data to be documented for the service provider for addressing the opportunity as per the defined KPIs. Then charter informed what provider has done and his next action to do based on the documentation that is done for the beneficiary's encounter/visit at step 212.

This is a two-way communication between a provider and a charter to ensure the opportunities that have been identified as addressed are re-run with the new data that was documented for the encounter/visit. If the opportunity remains, the system informs the provider of potential missing data or values that will address an opportunity. The charting can either be driven by a person (i.e. documenting data on paper) or by a system/software (for example, computer system or laptop or Microsoft office).

The process then moves to the stage of coding wherein at step 213 the coder ensures that all the care provided is coded. The coder compares the coded data with the data to be coded provided by the service provider or charter. The coder provides information about the work which has been done by the provider or charter etc. If the coder does not find out any evidence of the action taken by the service provider or charter, then coder requests for additional information from the service provider and charter and further suggests and generate inbuilt codes for the next action to be taken to ensure the KPIs are accurately addressed at step 213. There is also a two-way communication between the charter and the coder to ensure that all the documentation provided by the charter has been coded by the coder for proper addressing an opportunity. The coding can either be driven by a software or a combination of software or hardware. At step 214 the coder trying to address an opportunity based on the suggested code or other codes if the suggested codes being not able to address an opportunity. Intermediate opportunity was addressed based on the action taken by the coder at step 216. The intermediate opportunity that has been addressed is forwarded to the data analytics system for updating the data of the beneficiary. Further, at step 217 follow up or appointment of the beneficiary is required if the opportunities was not addressed at step 215. Those opportunities which have been addressed at step 215 are further updated in terms of claims or clinical data at step 216.

The updated information of the beneficiary received from step 216 is forwarded to the population information system 101 for updating the information (i.e. opportunity provided to the beneficiary has been addressed/not required follow-up or not). The updated information of the beneficiary received from step 216 is further forwarded to the clinical data of the payer system at step 303 which comprises information of the beneficiary health (i.e. GIC forms etc.) provided by the provider for billing. The updated information of the beneficiary received from step 216 is further forwarded to the claim data of the payer system.

Moving to the next step, it is made sure that all the completed claims or clinical data which are obtained from step 216 should be submitted to payer for billing.

The system/modules of payers will receive enriched claims data (i.e. clinical and financial data etc.) to the insurer for settlement and address opportunity at step 301. Submission of claims is a standard process in requesting and receiving payment in healthcare. In addition, clinical or supplemental data can be gathered to support the closure of opportunities. The system gathers the appropriate clinical information relating to the beneficiary from the received claims at step 303. The clinical data 303 and financial data 304 is forwarded to the data analytics system for upgrading the information of the beneficiary. Where the information does not exist in structured data, the system prompts the population information system (or the appropriate stakeholder) to enter the value of the missing clinical data to be captured and submits them to the insurer or appropriate stakeholders. The results generated after processing the clinical and financial data is updated to the data analytics system. If, however any new opportunity is identified in the claim or additional clinical data, the data relating to such identified opportunity is shared across healthcare network (i.e. providers or practice administrators) and the entire process discussed above is repeated to be address the identified opportunities.

The steps of the process are performed in response to a trigger event or as part of the standard workflow process of generating payment through the claims process, such as entry of information for one or more beneficiary into an electronic medical record. The steps are performed during a encounter/visit at a clinic, physician's office, hospital or other healthcare facility or during the normal processing of a claim which often happens after the beneficiary has completed the visit and left the physician's office or facility. By performing the steps of the process during an encounter/visitor within existing processes, the care of the beneficiary is tuned based on the most recent data in order to identify an opportunity. The care of the beneficiary is managed based on the current status of the beneficiary derived from beneficiary specific data and determination of any opportunity in the care.

In one embodiment, the act of coding includes that the data or information used in the process is translated to a unified coding. The mining from the different data sources with many possible different formats existing or new allows storage of the data into a database with a unified coding. A given format is used for the database. Clinical data extracted from different sources and in respective different formats is stored in a unified coding. One format is used for the data mined from many different formats. In one embodiment, the unified coding is of medical entities or concepts, attributes of the entities, and values of the attributes. Alternatively, other unified coding formats existing or new for the values may be used.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), an inter-network (e.g., the Internet), and peer-to-peer networks (e.g., ad hoc peer-to-peer networks).

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

The computing system can include clients and servers. A client and server are generally remote from each other and typically interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server. The computing system can use one or more client and server to process the data in the healthcare system.

Although, the present invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Thus, from the foregoing description, it will be apparent to one of ordinary skill in the art that many changes and modifications can be made thereto without departing from the spirit or scope of the invention. Practitioners of the art can derive several embodiments and domains of applicability of the present invention.

For example, in other embodiments, the process encompasses setting up an electronic visit or an e-visit for the beneficiary to communicate with the provider. An e-care summary sheet generated for the e-visit lists the opportunity in care identified, that can be discussed between the provider and the beneficiary to arrive at a remedial action plan. The obvious benefit of the e-visit is that the beneficiary does not need to physically visit the provider. The beneficiary can connect and communicate with the provider from his/her mobile device or personal computer or thorough emails, using electronic methods for meeting on the internet. In a modification of this embodiment, the e-visit may be substituted by a phone or mobile call

Accordingly, it is not intended that the scope of the foregoing description be limited to the exact description set forth above, but rather that such description be construed as encompassing such features that reside in the present invention, including all the features and embodiments that would be treated as equivalents thereof by those skilled in the relevant art.

It will also be understood that each block/circle of the diagram and/or flowchart illustrations, and combinations of blocks/circle in the diagrams and/or flowchart illustrations, can be implemented by special purpose hardware-based systems which perform the specified functions or acts, or combinations of special hardware and software instructions.

Thus, it is intended that the scope of the present invention herein disclosed should not be limited by the embodiments described above but should be determined only by a fair reading of the appended claims.

1. A method for collaboration between various stakeholders in a healthcare industry to correctly identify and compute key performance indicators (KPIs), and any opportunity, which increases performance on said KPIs communicated to all stakeholders ensuring identification, codification and completion of required services addressing said opportunity, the method comprising the steps of:
identifying beneficiary's data or information from various sources across a healthcare network (131);
defining key performance indicator(s) (KPI) for setting the rules to process the incorporated data or information of the beneficiary for identification of an opportunity (132);
computing the performance based on said KPIs (137) against the data collected of the beneficiary (131);
examining and identifying whether beneficiary requires any opportunity for improvement in healthcare services (133);
reviewing the identified opportunity and enabling appropriate stakeholders to engage with the beneficiary in the appropriate way to address the identified opportunity (201) for beneficiaries that require care, notifying the patients to schedule them either through direct outreach or through electronic notification so the beneficiary can schedule their own appointment (204/205)
reviewing the identified opportunities when the beneficiary comes for care during an encounter/visit of the beneficiary (206) and capturing that the opportunity has been cared (209), further care is needed (208), the care has already been provided or there is an exclusion or exception that means the care is not needed for the said beneficiary (No number on chart);
charting said captured information in a proper manner to address the identified opportunity as per the defined KPI to provide the care to the beneficiary (212);
coding the charted information in a proper manner to ensure that said opportunities as per the defined KPIs are addressed properly (213 and 214);
re-evaluating the captured information to determine whether the data created in the coding and charting completed the intent of the KPIs or a follow up visit/encounter is needed with beneficiary (217);
ascertaining the care provided and marking the KPIs and opportunities as addressed (215); and
updating the information of the beneficiary to the analytic system and payer system (216).

2. The method as claimed in claim 1, wherein the opportunity relates to the deviation in delivering care to the beneficiary wherein deviation includes a gap in clinical quality, risk assessment and/or stratification, diagnosis, care, screening, tests, treatments, assessment, documentation, waste avoidance, data accuracy and compliance thereof.

3. The method as claimed in claim 1, wherein the step of examining and identifying whether beneficiary requires further opportunity for improvement in healthcare services (133) comprising the steps of:
reviewing identified opportunity (135);
notifying an administrator of said beneficiary for scheduling appointments with a service provider (201);
sending scheduled information to the beneficiary (204);
capturing data or information of the beneficiary (205); and
providing care to the beneficiary on scheduled visit/encounter (206).

4. The method as claimed in claim 1, wherein the step of reviewing the identified opportunity during the encounter/visit of the beneficiary receiving care and capturing the information of said beneficiary (206) comprising the steps of:
capturing and displaying the opportunity that have to be addressed to care giver (207);
tracking the captured opportunity to be addressed against a workflow which tracks orders or tests and informs appropriate stakeholders whether the additional procedures, medications or tests performed or not performed in appropriate timeframes (208);

5. The method as claimed in claim 1, wherein steps of charting of said captured information in a proper manner to address the identified opportunity as per the defined KPIs (212) comprising the steps of:
comparing the data already documented with the data to be documented by the service provider for addressing the opportunity as per the defined KPIs; and
suggesting and generating next action, for one or more stakeholders, to be taken by the coder for adding data or the service provider for additional care for addressing the identified opportunity.

6. The method as claimed in claim 1, wherein steps of coding of the charted information in a proper manner to ensure that said opportunities as per the defined KPIs are addressed properly (213 and 214) comprising the steps of:
comparing coded data with the data to be coded provided by the service provider and/or charter;
identifying opportunity based on the action taken by service provider and/or charter etc;
requesting information from the service provider and charter for ensuring that KPIs are accurately addressed;
suggesting and generating inbuilt codes for the next action to be taken to ensure the KPIs are accurately addressed; and
addressing intermediate opportunity based on the action taken.

7. The method as claimed to claim 1, wherein the step of re-evaluating the captured information to determine whether the data created in the coding and charting completed the intent of the KPIs or a follow up visit/encounter is needed with beneficiary (217) comprising the steps of:
capturing and confirming all the information of charting and coding are completed to address the opportunity (214);
addressing and ascertaining the addressed opportunity which includes creating additional records to submit where the enriched claims do not support the ability to capture data needed to address the opportunity as a modification to existing enriched claims format, or adding an additional data exchange format to existing enriched claims format, or replacing the existing enriched claims format by some other data exchange (216); and
converting addressed opportunity into the enriched claims for compliance or billing (216).

8. The method as claimed in claim 1, wherein the step of updating the information of the beneficiary to the analytic system and payer system (216) comprising the steps of:
delivering enriched claims to the payers for billing (301);
leveraging the data from enriched claim to update data in analytics system (131)
calculating the KPI based on the enriched claim which contains the information included from the addressed opportunity (137); and
checking compliance based on the particular KPI (134).

9. The method as claimed in claim 1, wherein the data and/or information of the beneficiary is obtained, updated from and updated to various sources includes, beneficiary's clinical data, public health data, electronic health record and scheduling system, enriched claim, payers, service providers, laboratories, imaging and hospital systems.

10. The method as claimed in claim 1, wherein the stakeholder in the healthcare industry including but not limited to, hospitals, doctors, insurance companies, lab companies, nursing homes, or any other entity involved in care of the beneficiary or the like.

11. A system for collaboration between various stakeholders in a healthcare industry to correctly identify and compute key performance indicators (KPIs), and any opportunity, which increases performance on the said KPIs communicated to all stakeholders ensuring identification, codification and completion of required services addressing of said opportunity, the system comprising:
one or more data analytics server;
one or more central database server;
one or more service provider server;
one or more mobile computing device;
one or more software, with or without a graphical user interface, to define KPIs;
one or more metrics engine for calculating KPIs;
one or more workflow engine for determining the right interaction between stakeholders;
one or more graphical user interface to display information to and take input from end users of the system;
one or more payer server associated with the healthcare network system;
one or more electronically storage medium for storing healthcare records; and
wherein said system configured to perform operations comprising:
identifying, information of the beneficiary from the central database server (131);
compiling, data received from the database server by the data analytics server (131);
defining, key performance indicator (KPI) for setting the rules to process the incorporated data or information of the beneficiary for identification of an opportunity (132);
computing the performance based on said KPIs (137);
examining and identifying at data analytics server, whether beneficiary requires any opportunity for improvement in healthcare services (133);
reviewing by the service provider, the identified opportunity and enabling appropriate stakeholders to engage with the beneficiary in the appropriate way to address the identified opportunity (201) for beneficiaries that require care, notifying the patients to schedule them either through direct outreach or through electronic notification so the beneficiary can schedule their own appointment (204/205);
reviewing the identified opportunities when the beneficiary comes for care during an encounter/visit of the beneficiary (206) and capturing the opportunity has been cared (209), further care is needed (208), the care has already been provided or there is an exclusion or exception that means the care is not needed for the said beneficiary (No number on chart);
charting by the charter, said captured information in a proper manner to address the identified opportunity as per the defined KPI to provide the care to the beneficiary (212);
coding by the coder, the charted information in a proper manner to ensure that said opportunities as per the defined KPI are addressed properly (213 and 214);
re-evaluating by the service provider server, the captured information to determine whether the data created in the coding and charting completed the intent of the KPIs or a follow up visit/encounter is needed with beneficiary (217);
ascertaining by the service provider server, the care provided and marking the KPIs and opportunities as addressed (215); and
updating by the service provider server, information of the beneficiary to the data analytic server and payer server (216).

12. The system as claimed in claim 11, wherein the analytics server examines (133), whether beneficiary requires further opportunity for improvement in healthcare services and configured to perform operations comprising
reviewing by the analytics system, identified opportunity (135);
notifying by provider system, to the administrator of said beneficiary for scheduling appointments with a service provider (201);
sending by mobile computing device or email or SMS or notification being updated on a portal, scheduled information to the beneficiary (204);
capturing by the service provider server, data and information of the beneficiary (205); and
providing by the service provider, care to the beneficiary on scheduled visit/encounter (206).

13. The system as claimed in claim 11, wherein the service provider review, the identified opportunity during the encounter/visit of the beneficiary receiving care and capturing the information of said beneficiary (206) and configured to perform operations comprising
capturing the opportunity that have to be addressed (207); and
tracking the captured opportunity to be addressed against a workflow which tracks orders or tests and informs appropriate stakeholders whether the additional procedures, medications or tests performed or not performed in appropriate timeframes (208).

14. The system as claimed in claim 11, wherein the charter charting (212), said captured information in a proper manner to address the identified opportunity as per the defined KPIs (212) and configured to perform operations comprising
comparing the data already documented by the data provided by the service provider for addressing the opportunity as per the defined KPIs; and
suggesting and generating by the service provider, next action to be taken by the coder for adding data or the service provider for additional care for providing opportunity.

15. The system as claimed in claim 11, wherein the coder coding, the charted information in a proper manner to ensure that said opportunities as per the defined in the KPIs (213 and 214) and configured to perform operations comprising comparing coded data with the data which marked as addressed by the charter and/or provider;
identifying opportunity based on the action taken by service provider or charter etc;
requesting information from the service provider and charter for ensuring that KPIs are accurately addressed;
suggesting and generating inbuilt codes for the next action taken to be taken to ensure the KPIs are accurately addressed; and
addressing intermediate opportunity based on the action taken.

16. The system as claimed to claim 11, wherein the service provider server re-evaluate, the captured information to determine whether the data created in the coding and charting completed the intent of the KPIs (217) or if a follow up visit/encounter is needed with beneficiary (217) and configured to perform operations comprising capturing and confirming all the information of charting and coding are completed to address the opportunity (214);
addressing and ascertaining the addressed opportunity includes creating additional records to submit where the enriched claims does not support the ability to capture data needed to address the opportunity as a modification to existing enriched claims format, or adding an additional data exchange format to existing enriched claims format, or replacing the existing enriched claims format by some other data exchange format (215); and
converting provided opportunity into the enriched claims for compliance or billing (216).

17. The system as claimed in claim 11, wherein the service provider server update, information of the beneficiary to the data analytic system and payer system (216) and configured to perform operations comprising
delivering enriched claims to the payers for billing (301);
leveraging the data from enriched claim to update data in analytics system (131)
calculating the KPI based on the enriched claim which contains the information included from the addressed opportunity (137); and
filing compliance based on the particular KPI (134).
